(19) United States
(12) Patent Application Publication
(10) Pub. No.: US 2023/0383314 A1
(43) Pub. Date: Nov. 30, 2023

(54) Olivetolic Acid Cyclases for Cannabinoid Biosynthesis

(71) Applicant: CB Therapeutics, Inc., Carlsbad, CA (US)

(72) Inventors: Erin Marie Scott, Encinitas, CA (US); Jacob Michael Vogan, San Diego, CA (US); Tyler Huelsman, San Diego, CA (US)

(73) Assignee: CB Therapeutics, Inc., Carlsbad, CA (US)

(21) Appl. No.: 17/827,640

(22) Filed: May 27, 2022

(51) Int. Cl. C12N 15/90; C12N 15/81; C12N 15/52; C12N 9/12

(52) U.S. Cl. CPC C12N 15/905; C12Y 404/01026; C12N 15/81; C12N 15/52; C12N 9/12

(57) ABSTRACT

Provided is an engineered olivetolic acid cyclase (OAC) enzyme derived from a microorganism or non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃

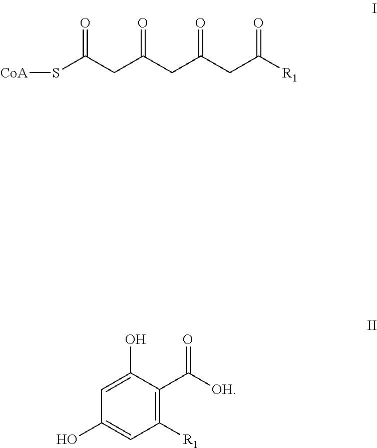

Also provided is nucleic acids encoding the above OAC enzyme, expression cassettes comprising those nucleic acids, and recombinant microorganisms comprising those expression cassettes that express the OAC enzyme encoded therein.

Additionally provided is a method of converting a polyketide (I) into a resorcyclic acid derivative (II) comprising contacting the polyketide with the above-identified OAC enzyme in a manner and for a time sufficient to convert the polyketide (I) into the resorcyclic acid derivative (II).

### RELATED APPLICATIONS

The present application claims priority to U.S. provisional patent application No. 63/194,121, filed on May 27, 2021, the disclosure and content of which is incorporated herein by reference in its entirety for all purposes.

### SEQUENCE LISTING ON ASCII TEXT

This patent application file contains a Sequence Listing submitted in computer readable ASCII text format (file name: CBTH-13-US_SeqList.txt, date recorded: May 27, 2022, size: 1,485,254 bytes). The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The content of the Sequence Listing file is incorporated herein by reference in its entirety.

### TECHNICAL FIELD

The present application generally relates to recombinant enzymes and genes encoding those enzymes. More specifically, the application provides recombinant olivetolic acid cyclase genes and enzymes that function in microorganisms.

### BACKGROUND

Cannabinoids are a class of organic small molecules of meroterpenoid structures found in the plant genus Cannabis. The small molecules are currently under investigation as therapeutic agents for a wide variety of health issues, including epilepsy, pain, and other neurological problems, and mental health conditions such as depression, PTSD, opioid addiction, and alcoholism (Committee on the Health Effects of Marijuana, 2017).

While it is known that cannabinoids may be obtained via biosynthesis in plant species, there are many problems associated with the synthesis of such molecules which need to be overcome, including problems with large-scale manufacturing, purification, and heterologous expression for biosynthesis.

Producing cannabinoids, in recombinant microorganisms such as yeast is a promising solution to the above problems. See, e.g., U.S. patent application Ser. Nos. 16/553,103, 16/553,120, 16/558,973, 17/068,636 and 63/053,539; U.S. Pat. No. 10,435,727; and US Patent Publications 2020/0063170 and 2020/0063171, all incorporated by reference.

One way to improve biosynthetic cannabinoid production in microorganisms is by the discovery and use of new enzymes that catalyze the same reactions as plant derived enzymes but with improved parameters. The present invention provides such enzymes with olivetolic acid cyclase activity.

In Cannabis spp., the olivetolic acid cyclase (OAC) enzyme catalyzes the cyclization of the linear polyketide olivetol to form olivetolic acid (OA), a precursor for biosynthesis of downstream cannabinoids such as cannabidiol (CBD) and tetrahydrocannabinol (THC). See FIG. 1. OAC can also form many variants of olivetolic acid, by accepting polyketides with longer or shorter alkyl chains (FIG. 1, R1 group). Examples include varinolic or divariniolic acid, orsellinic acid, phorolic or sphaerophorolic acid. Characterization of the enzyme responsible for this reaction in the Cannabis plant was published (Gagne, 2012) and the enzyme was shown to be a member of the DABB domain protein family. The crystal structure of OAC was published in 2016 (Yang, 2016) and the mechanism of the reaction characterized as a C2-C7 aldol cyclization.

DABB domains are small alpha/beta barrel motifs of unknown function. They are known to be upregulated in response to salt stress in plants and for purposes of molybdopterin uptake. A description of the domain can be found in the SMART (Simple Modular Architecture Research Tool) database (Letunic I, Khedkar S, Bork P. SMART: recent updates, new developments and status in 2020. Nucleic Acids Res. 2021 Jan. 8; 49(D1):D458-D460). The domain typically forms an alpha-beta barrel dimer.

There remains a need for new engineered enzymes that are capable of catalyzing the cyclization of the linear polyketide olivetol to form olivetolic acid (OA), which is a precursor for biosynthesis of downstream valuable cannabinoid molecules.

### SUMMARY

Engineered enzymes from microorganisms with OAC activity are provided herein.

Provided is an engineered olivetolic acid cyclase (OAC or altOAC or PKSC) enzyme derived from a microorganism or a non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃

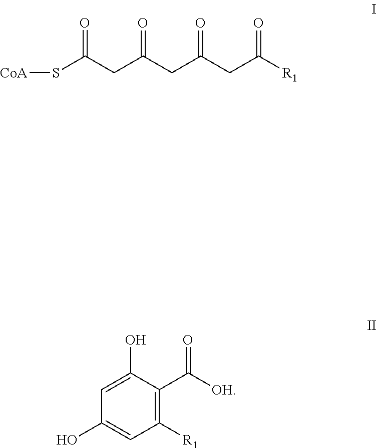

Also provided is engineered nucleic acids encoding the above OAC enzyme, expression cassettes comprising those nucleic acids, and recombinant microorganisms comprising those expression cassettes that express the OAC enzyme encoded therein.

Additionally provided is a method of converting a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃,

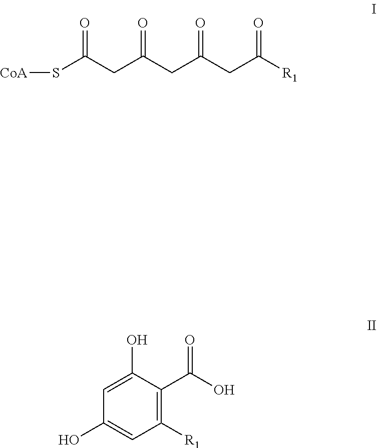

the method comprising contacting the polyketide with the above-identified engineered OAC enzyme in a manner and for a time sufficient to convert the polyketide (I) into the resorcyclic acid derivative (II).

The present teachings also include an engineered olivetolic acid cyclase (OAC or altOAC or PKSC) enzyme derived from a microorganism or a non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃

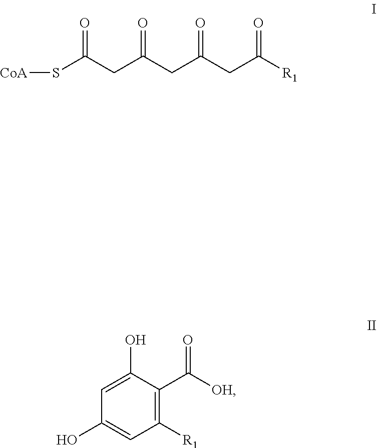

and wherein the enzyme comprises an amino acid sequence that is at least 95% identical to any one of the sequences set forth in SEQ ID NO: 193-SEQ ID NO: 378. In some preferred embodiments, the enzyme comprises an amino acid sequence that is at least 99% identical to any one of the sequences set forth in SEQ ID NO: 193-SEQ ID NO: 378. In some more preferred embodiments, the enzyme comprises an amino acid sequence that is one of the sequences set forth in SEQ ID NO: 193-SEQ ID NO: 378. In some preferred embodiments, the enzyme comprises an amino acid sequence that is at least 50% identical to the sequence set forth in either SEQ ID NO: 385 or SEQ ID NO: 386. In some embodiments, the enzyme comprises an amino acid sequence that is at least 20% identical to SEQ ID NO: 334.

The present teachings also include an isolated codon-optimized polynucleotide that encodes the above-identified engineered OAC enzymes, comprising a nucleotide sequence that is at least 50% identical to the sequence set forth in either SEQ ID NO:1, or SEQ ID NO:2, or SEQ ID NO:3. In some embodiments, the codon-optimized polynucleotide that encodes the above-identified engineered OAC enzymes comprises a nucleotide sequence that is at least 95% identical to any one of the sequences set forth in SEQ ID NO: 1-SEQ ID NO: 186. In some embodiments, the codon-optimized polynucleotide that encodes the above-identified engineered OAC enzymes comprises a nucleotide sequence that is at least 95% identical to sequence set forth in SEQ ID NO: 142. In some embodiments, the codon-optimized polynucleotide is inserted in a vector configured for replication and protein expression in microbial (e.g., yeast) cells.

It is expected that the above-identified engineered OAC enzymes from a microorganism would have low amino acid sequence homology (e.g., less than 60%, less than 50%, less than 40%, less than 30% or less than 20% sequence homology) to amino acid sequences of OAC from a Cannabis sp (for example, to sequence set forth in SEQ ID NO: 379).

### BRIEF DESCRIPTION OF THE DRAWINGS

FIG. 1A depicts a generalized cannabinoid biosynthesis pathway, including structures of cannabinoid molecules and variants.

FIG. 1B depicts an olivetolic acid-specific cannabinoid biosynthesis pathway.

FIG. 2 depicts condensation reaction mechanisms which could result in the formation of olivetolic acid from olivetol, including C2-C7 intramolecular aldol, Diekmann condensation, Claisen condensation and Knoenvagel condensation.

FIG. 3 depicts the domain architecture of a polyketide synthase enzyme (PKS) including domains which might function as independent cyclase enzymes.

FIG. 4 shows graphs of HPLC data showing olivetolic acid production in vivo by altOAC 75 with amino acid sequence set forth in SEQ ID NO: 334.

FIG. 5 shows an exemplary clustermap comparing selected altOAC enzymes. The value in each cell is the percentage of identical amino acid residues normalized by the ratio of alignment length to sequence length, computed with BLAST (Basic Local Alignment Search Tool, NCBI).

FIG. 6 shows relative amount of CBGA produced in recombinant yeast cells capable of generating downstream cannabinoids depending on the OAC genes expressed in these cells. It depicts the yield of the cannabinoid, cannabigerolic acid (CBGA), via expression of an optimized cannabis olivetolic acid cyclase (csOAC) alone (SEQ ID NO: 187), or in combination with other OAC enzyme constructs in the recombinant host expressing downstream cannabinoid enzymes.

### DETAILED DESCRIPTION OF EMBODIMENTS

### Abbreviations and Definitions

To facilitate understanding of the invention, a number of terms and abbreviations as used herein are defined below as follows:

Conservative amino acid substitutions: As used herein, when referring to mutations in a protein, “conservative amino acid substitutions” are those in which at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional variant: The term “functional variant,” as used herein, refers to a recombinant enzyme such as a OAC enzyme that comprises a nucleotide and/or amino acid sequence that is altered by one or more nucleotides and/or amino acids compared to the nucleotide and/or amino acid sequences of the parent protein and that is still capable of performing an enzymatic function (e.g., synthesis of olivetolic acid) of the parent enzyme. In other words, the modifications in the amino acid and/or nucleotide sequence of the parent enzyme may cause desirable changes in reaction parameters without altering fundamental enzymatic function encoded by the nucleotide sequence or containing the amino acid sequence. The functional variant may have conservative change including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids. Also envisioned is the use of amino acid analogs, e.g. amino acids not DNA or RNA encoded in biological systems, and labels such as fluorescent dyes, radioactive elements, electron dense agents, or any other protein modification, now known or later discovered.

The term “modified” or “engineered”, or “variant”, as used in reference to nucleic acid molecules and protein molecules, e.g., an engineered enzyme, implies that such molecules are created by human intervention and they are non-naturally occurring. The engineered enzyme is a polypeptide or peptide having an altered amino acid sequence, relative to an unmodified or wild-type protein, such as starting naturally occurring enzyme (wild-type enzyme), or a portion thereof. An engineered enzyme is a polypeptide or peptide which differs from a wild-type enzyme sequence, or a portion thereof, by one or more amino acid substitutions, deletions, additions, or combinations thereof. Sequence of an engineered enzyme can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences (e.g., mutations) compared to the sequence of starting wild-type enzyme. An engineered enzyme generally exhibits at least 70%, 80%, 85%, 86%, 87%, 88%, 89%, 90%, 91%, 92%, 93%, 94%, 95%, 96%, 97%, 98%, 99% or more sequence identity to a corresponding starting wild-type enzyme. An engineered enzyme can exhibit at least 70%, 80%, 85%, 86%, 87%, 88%, 89%, 90%, 91%, 92%, 93%, 94%, 95%, 96%, 97%, 98%, 99% or more sequence homology to a corresponding starting wild-type enzyme. Non-naturally occurring amino acids as well as naturally occurring amino acids are included within the scope of permissible substitutions or additions. An engineered enzyme is not limited to any enzymes made or generated by a particular method of making and includes, for example, an engineered enzyme made or generated by genetic selection, protein engineering, directed evolution, de novo recombinant DNA techniques, or combinations thereof.

In some embodiments, variants of an engineered enzyme displaying only non-substantial or negligible differences in structure can be generated by making conservative amino acid substitutions in the engineered enzyme. By doing this, engineered enzyme variants that comprise a sequence having at least 90% (90%, 91%, 92%, 93%, 94%, 95%, 96%, 97%, 98%, and 99%) sequence identity with wild-type or another engineered enzyme sequences can be generated, retaining at least one functional activity of the engineered enzyme, e.g., ability to catalyze a specific reaction. Examples of conservative amino acid changes are known in the art. Examples of non-conservative amino acid changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histidine, for (or by) an electronegative residue, e.g., glutamic acid or aspartic acid; or (d) a residue having a bulky side chain, e.g., phenylalanine, for (or by) one not having a side chain, e.g., glycine. Methods of making targeted amino acid substitutions, deletions, truncations, and insertions are generally known in the art. For example, amino acid sequence variants can be prepared by mutations in the DNA. Methods for polynucleotide alterations are well known in the art, for example, Kunkel et al. (1987) Methods in Enzymol. 154:367-382; U.S. Pat. No. 4,873,192 and the references cited therein.

The term “sequence identity” as used herein refers to the sequence identity between polynucleotides or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. Methods for the alignment of sequences for comparison are well known in the art, such methods include the BLAST algorithm, which calculates percent sequence identity and performs a statistical analysis of the similarity between the two sequences. The software for performing BLAST analysis is publicly available through the National Center for Biotechnology Information (NCBI) website.

The term “sequence homology” as used herein is a measure of similarity and refers to the sequence similarity between polynucleotides or proteins at the nucleotide or amino acid level, respectively. The protein sequence homology may be determined by comparing the amino acid sequence in a given position in each sequence when the sequences are aligned. Similarly, the nucleic acid sequence homology may be determined by comparing the nucleotide sequence in a given position in each sequence when the sequences are aligned. “Sequence homology” means the percentage of homologous subunits (i.e., amino acids) at corresponding positions in two sequences when the two sequences are aligned to maximize subunit matching, i.e., taking into account gaps which factor in insertions and deletions in the aligned sequences. Sequence homology is present when a subunit position in each of the two or more sequences is occupied by identical amino acid residues or functionally similar amino acid residues (e.g., isosteric or isoelectric amino acid identities; amino acid residues that belong to the same functional class, such as e.g. positively charged residues, or small hydrophobic residues). Sequence homology is absent when a subunit position in each of the two or more sequences is occupied by a functionally different amino acid (i.e., lacking structural and/or functional similarity). Methods for the alignment of sequences for comparison are well known in the art, such methods include the BLAST algorithm, which calculates percent sequence homology and performs a statistical analysis of the homology between the two sequences.

The terms “corresponding to position(s)” or “position(s) . . . with reference to position(s)” of or within a peptide or a polynucleotide, such as recitation that nucleotides or amino acid positions “correspond to” nucleotides or amino acid positions of a disclosed sequence, such sequence set forth in the Sequence Listing, refers to nucleotides or amino acid positions identified in the polynucleotide or in the peptide upon alignment with the disclosed sequence using a standard alignment algorithm, such as the BLAST algorithm (NCBI). One skilled in the art can identify any given amino acid residue in a given peptide at a position corresponding to a particular position of a reference sequence, such as set forth in the Sequence Listing, by performing alignment of the peptide sequence with the reference sequence (for example, by using BLASTP publicly available through the NCBI website), matching the corresponding position of the reference sequence with the position in peptide sequence and thus identifying the amino acid residue within the peptide. Amino acid positions corresponding to the recited residues can be also determined by structural alignment to the experimentally-determined template structure in the PDB (as given by the PDB accession code after making structural truncations corresponding to the SEQ ID NO of interest). The reference structures used in the structural alignment can be experimentally determined or generated by homology modeling using state of the art homology modeling methods such as Rosetta or PyRosetta macromolecular software suites, machine learning models such as AlphaFold2, or the like. Other useful structural alignment methods and/or programs include, but are not limited to, TM-align, PyMOL (superalign, cealign, and align methods), LSQMAN, Fr-TM-align, DALI, DaliLite, CE, CE-MC, and the like.

Recombinant nucleic acid and recombinant protein: As used herein, a recombinant nucleic acid or protein is a nucleic acid or protein produced by recombinant DNA technology, e.g., as described in Green and Sambrook (2012).

Polypeptide, protein, and peptide: The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Primer, probe and oligonucleotide: The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. In certain aspects, they are of a length of from about 8 nucleotides to about 200 nucleotides. In other aspects, they are from about 12 nucleotides to about 100 nucleotides. In additional aspects, they are about 18 to about 50 nucleotides. They can be labeled with detectable markers or modified in any conventional manners for various molecular biological applications.

Vector: As used herein, the term “vector” refers to a nucleic acid molecule capable of transporting another nucleic acid to which it has been linked. One type of vector is an episome, i.e., a nucleic acid capable of extra-chromosomal replication. Various vectors are those capable of autonomous replication and/expression of nucleic acids to which they are linked. Vectors capable of directing the expression of genes to which they are operatively linked are referred to herein as “expression vectors.”

Linker: The term “linker” refers to a short amino acid sequence that separates multiple domains of a polypeptide. In some embodiments, the linker prohibits energetically or structurally unfavorable interactions between the discrete domains.

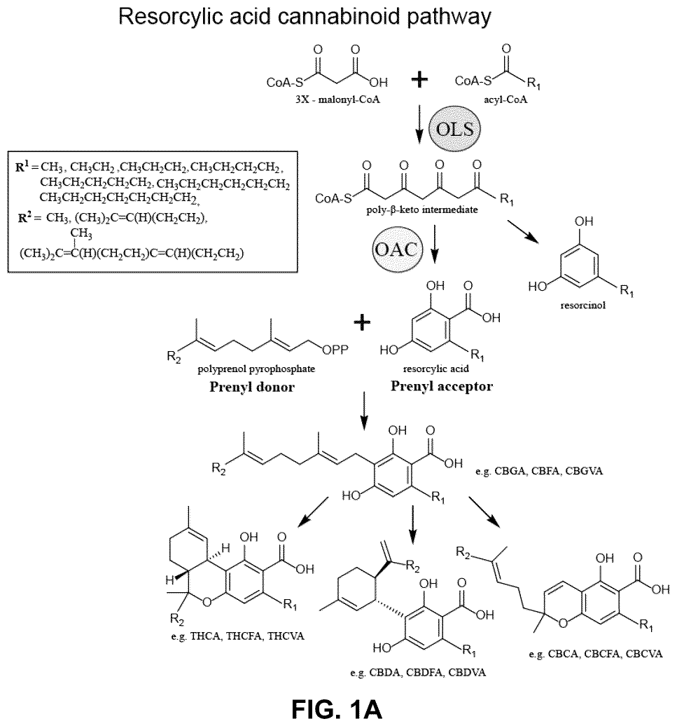

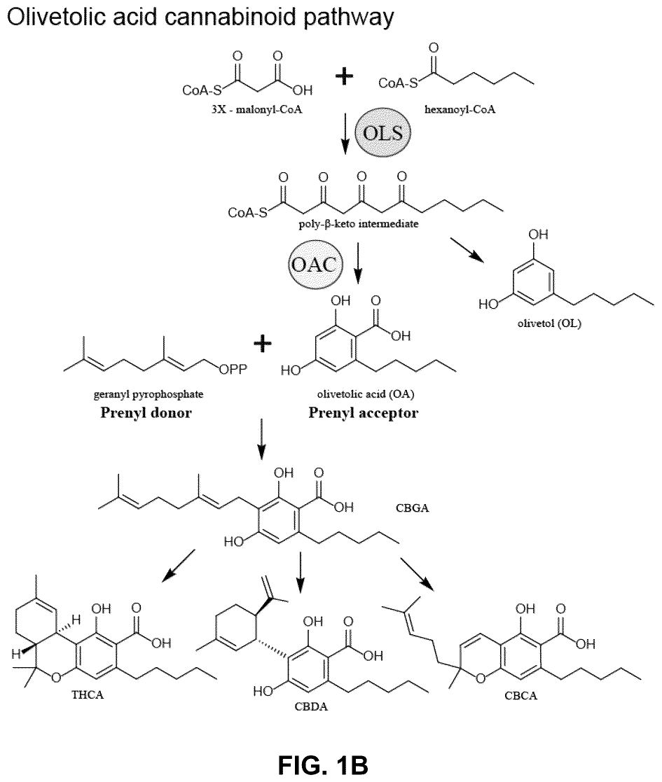

Cannabinoid: As used herein, the term “cannabinoid” refers to a family of structurally related aromatic meroterpenoid molecules. Cannabinoids are generally formed by the enzymatic fusion, by a cannabinoid synthase (having geranylpyrophosphate:olivetolate geranyltransferase activity), of an alkylresorcylic acid

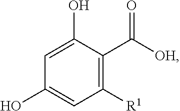

where R₁=CH₃, (CH₂)₂CH₃ (divarinolic acid), (CH₂)₄CH₃ (olivetolic acid), or (CH₂)₆CH₃, with a polyprenyl pyrophosphate such as geranyl pyrophosphate, neryl pyrophosphate, geranylgeranyl pyrophosphate, of farnesyl pyrophosphate (FIG. 1; see also Luo et al., 2019; Carvalho et al., 2017; and Gülck and Møller, 2020 and references cited therein). The polyprenyl pyrophosphate is synthesized by geranyl pyrophosphate synthase (GPPS) (U.S. Provisional Patent Application 63/141,486).

Codon optimized: As used herein, a recombinant gene is “codon optimized” when its nucleotide sequence is modified to accommodate codon bias of the host organism to improve gene expression and increase translational efficiency of the gene.

Expression cassette: As used herein, an “expression cassette” is a nucleic acid that comprises a gene and a regulatory sequence operatively coupled to the gene such that the promoter drives the expression of the gene in a cell. An example is a gene for an enzyme with a promoter functional in yeast, where the promoter is situated such that the promoter drives the expression of the enzyme in a yeast cell.

Olivetolic Acid Cyclase Enzymes from Microorganisms and Non-Cannabis Plants

Provided herein is an olivetolic acid cyclase (OAC or altOAC or PKSC) enzyme derived from a microorganism or a non-Cannabis plant, where the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃

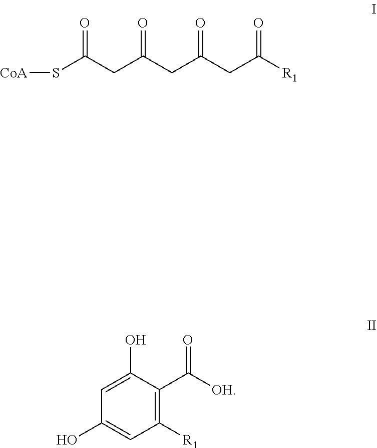

Many enzymes in nature catalyze cyclization reactions of polyketides. The ability of the plant enzyme to make OAC is not unique; enzymes derived from other species are able to catalyze the same reaction if the substrate is present.

It is useful to have several possible enzymes to catalyze a single step in any biosynthesis pathway. A new enzyme may have better catalytic properties than the original enzyme, may be higher throughput or have better binding affinity for the substrate. Other properties can make an alternative enzyme desirable as well, including better expression in a heterologous host and greater robustness under fermentation conditions, or requirements that more closely match the requirements of other enzymes in the pathway. There is also a benefit to using multiple OAC enzymes in combination within a single host in order to take advantage of different properties of the enzymes. Some variant enzymes may have longer half-lives inside the host, and so would be able to sustain high rates of catalysis for a longer time period. An additional desirable property would be the ability to selectively make one of the alkyl chain variant cannabinoids depicted in FIG. 1A and FIG. 1B.

In some embodiments, the enzyme is engineered by selected conservative or non-conservative amino acid changes to alter its substrate specificity making them more or less likely to synthesize a specific alkyl chain variant. Other enzymes are able to synthesize multiple variants. Enzymes with increased specificity for only one chain length are desirable for industrial purposes as a pure substrate leads to a pure product and eliminates the need to separate different but closely related variants after the fermentation.

These enzymes may be sourced from nature or specificity may be engineered into an enzyme by changing some residues, resulting in a non-natural amino acid sequence with improved properties. An example of this is the Y24F mutation described in Yang (2016) that increases the capacity of the Cannabis OAC for making OA, and also increases its specificity for OA over the lactone byproduct.

Enzyme variations may also result from mutations introduced into the DNA and amino acid sequences to prevent or promote post translational modifications of the protein. Nonlimiting examples of post translational modifications include phosphorylation, acetylation, methylation, SUMOylation, ubiquitination, proteolytic cleavage, lipidation, including prenylation such as farnesylation or myristoylation, glycosylation, nitrosylation and biotinylation.

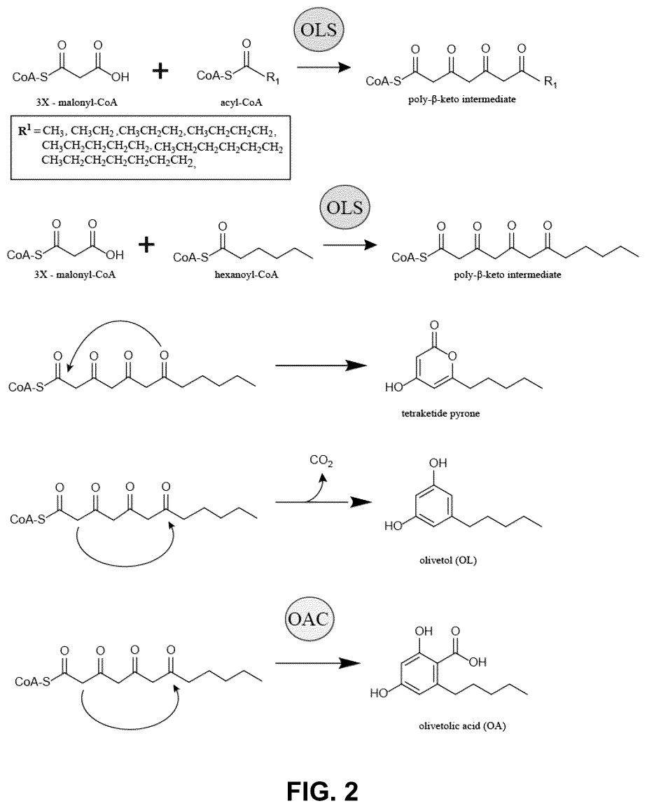

In some embodiments, the naturally occurring enzymes found in nature from non-Cannabis sources (e.g., a microorganism or a non-Cannabis plant) carries out the cyclization reaction using the same mechanism as the plant enzyme, i.e., a C2-C7 aldol cyclization. In other embodiments, the enzyme carries out the polyketide cyclization is by a different mechanism, for example Diekmann condensation, Claisen condensation or Knoevenagel condensation (FIG. 2).

Enzymes may be sourced from nature by homology to the plant enzyme, resulting in mostly DABB domain or DABB domain-like proteins. The OAC enzyme from Cannabis acts as a dimer. Novel altOAC or PKSC enzymes from other organisms may also act as dimer, though they need not necessarily dimerize in order to be active. They have potential to act as monomers or as higher order complexes, binding either other OACs or other proteins.

Enzymes that catalyze this type of reaction in fungi and bacteria are often involved in secondary metabolite biosynthesis. Genes involved in biosynthesis of a single compound are often organized into clusters in the genomes of fungi and bacteria, so searching genomes for secondary metabolite clusters is a way to uncover desirable candidate genes. Thus, in some embodiments, the altOAC or PKSC is encoded by a gene that is located within a secondary metabolite cluster in a microorganism.

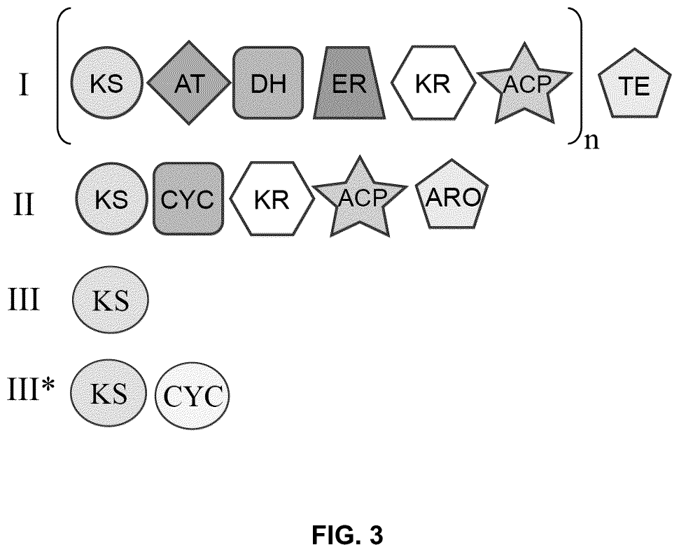

Another source of cyclases is individual domains of fungal polyketide synthases (PKSs). Polyketides are the basis for the biosynthesis of many secondary metabolites. Polyketide synthases (PKSs) are the enzymes that biosynthesize polyketides and these enzymes are conserved in multiple kingdoms. PKSs are particularly common in bacteria, plants and fungi. PKS enzymes are classified into three types (FIG. 3). Type I PKS are large multifunctional multidomain proteins. The number and arrangement of the domains is varied and contributes to the diversity of molecules synthesized. Type II PKSs are multienzyme complexes with a single set of iterative activities. Type III PKSs are smaller, homodimers of condensing enzymes. See Shen, 2003 for review of topic. Due to greater domain diversity, a domain capable of cyclizing OL to form OA is most likely to reside in a type I PKS, but could also be found in in a Type II or Type III PKS. Polyketide synthases catalyze the first committed step in cannabinoid biosynthesis, an assembly of a linear polyketide form short chain fatty acyl CoA precursors such as malonyl CoA and hexanoyl CoA. See Herbst, 2018 for review. The type of PKSs most often found in fungi are modular proteins that both assemble the polyketide and cyclize it (FIG. 3). Those PKSs therefore contain at least one cyclase domain (e.g., III* in FIG. 3). PKS enzymes that synthesize multicyclic structures often contain multiple domains with cyclase activity. Expression of the relevant cyclase domain from a fungal PKS alone enable cyclization of the linear polyketide to form OA. The most likely domains to independently catalyze cyclization are the PT (product template) domain and the TE-CLC domains. TE-CLC domains have thioesterase (TE) activity and also catalyze cyclization by an intramolecular Claisen condensation mechanism. They are also called Claisen cyclase domains (CLC)s reflecting this activity. Multiples of these domains may be found within a given PKS enzyme. PT domains, reviewed in Herbst, 2018 may also have elements of KS_DH domains.

It is expected that engineered altOAC or PKSC enzyme from a microorganism would have low amino acid sequence homology (e.g., less than 60%, less than 50%, less than 40%, less than 30% or less than 20% sequence homology) to amino acid sequences of OAC from a Cannabis sp (for example, to sequence set forth in SEQ ID NO: 379). In some preferred embodiments, the engineered olivetolic acid cyclase enzyme has an amino acid sequence that is less than 60% homologous to sequence set forth in SEQ ID NO: 334. In some preferred embodiments, the engineered olivetolic acid cyclase enzyme has an amino acid sequence that is less than 50%, less than 40%, less than 30% or less than 20% homologous to sequence set forth in SEQ ID NO: 334.

The present teachings include an engineered olivetolic acid cyclase (OAC or altOAC or PKSC) enzyme derived from a microorganism or a non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃

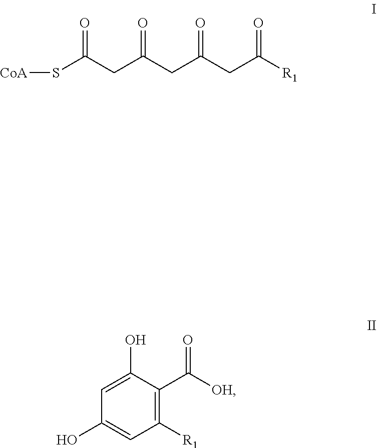

and wherein the enzyme comprises an amino acid sequence that is at least 90% or at least 95% identical, or at least 99% identical to any one of the sequences set forth in SEQ ID NO: 193-SEQ ID NO: 378. In some preferred embodiments, the enzyme comprises an amino acid sequence that is one of the sequences set forth in SEQ ID NO: 193-SEQ ID NO: 378. In some embodiments, the enzyme comprises an amino acid sequence that is at least 20%, at least 30%, at least 40%, at least 50%, at least 60%, at least 70%, at least 80%, or at least 90% identical to SEQ ID NO: 334. In some embodiments, the enzyme comprises an amino acid sequence that is at least 50%, at least 60%, at least 70%, at least 80%, or at least 90% identical to the sequence set forth in SEQ ID NO: 193, or in SEQ ID NO: 194, or in SEQ ID NO: 195.

The present teachings also include an isolated codon-optimized polynucleotide that encodes the above-identified engineered OAC enzymes, comprising a nucleotide sequence that is at least 50%, at least 60%, at least 70%, at least 80%, or at least 90% identical to the sequence set forth in SEQ ID NO: 1, or in SEQ ID NO: 2, or in SEQ ID NO: 3. In some embodiments, the codon-optimized polynucleotide that encodes the above-identified engineered OAC enzymes comprises a nucleotide sequence that is at least 90%, at least 95%, or at least 99% identical to any one of the sequences set forth in SEQ ID NO: 1-SEQ ID NO: 186. In some embodiments, the codon-optimized polynucleotide that encodes the above-identified engineered OAC enzymes comprises a nucleotide sequence that is at least 50%, at least 60%, at least 70%, at least 80%, at least 90%, or at least 95% identical to SEQ ID NO: 142. In some embodiments, the codon-optimized polynucleotide is inserted in a vector configured for replication and protein expression in microbial (e.g., yeast) cells.

In some embodiments, engineered olivetolic acid cyclase enzyme (e.g., PKSC) sequences listed in Table 1 and in the Sequence Listing are defined as (i) having at least 50% sequence identity to a reference sequence and (ii) functioning as part of a large, multiprotein complex. In some embodiments, engineered PKSC sequences, such as comprising amino acid sequence set forth in SEQ ID NOs: 196, 199, 202, 205, 208, 211, 214, 217, 220, 223, 235, 238 or 250, have at least 50% sequence identity to SEQ ID NO: 193. In other embodiments, engineered PKSC sequences, such as comprising amino acid sequence set forth in SEQ ID NOs: 197, 203, 206, 209, 212, 215, 218, 221, 224, 236, 251, 256 or 257, have at least 50% sequence identity to SEQ ID NO: 194. In yet other embodiments, engineered PKSC sequences, such as comprising amino acid sequence set forth in SEQ ID NOs: 204, 207, 210, 213, 216, 219, 222, 237, 255, 258, 259, have at least 50% sequence identity to SEQ ID NO: 195. In yet other embodiments, engineered olivetolic acid cyclase enzyme (e.g., altOAC) sequences listed in Table 1 and in the Sequence Listing comprise an amino acid sequence that is at least 50% identical to the sequence set forth in either SEQ ID NO: 385 or SEQ ID NO: 386. In yet other embodiments, engineered olivetolic acid cyclase enzyme (e.g., altOAC) sequences listed in Table 1 and in the Sequence Listing comprise an amino acid sequence that is at least 50% identical to the sequence set forth in SEQ ID NO: 334.

AltOAC sequences (for example, SEQ ID NO: 260-SEQ ID NO: 378) are characterized by greater sequence diversity, smaller protein size and the ability to act independently of non-OAC polypeptides. OACs also frequently contain a DABB domain, a protein motif that is associated with stress response. They share this in common with the OAC enzyme from Cannabis sativa and as a result may have some homology to csOAC. Similarities in amino acid sequences underlie similarities in protein function for the claimed engineered olivetolic acid cyclase enzyme. In some embodiments, altOAC enzymes contain a DABB domain, which is a structural motif that is often present in proteins involved in stress response in plants. Two sequences of DABB domain are provided herein (SEQ ID NO: 385-SEQ ID NO: 386), which can be used as reference sequences during aligning of sequences of altOAC enzymes disclosed herein. In some preferred embodiments, engineered olivetolic acid cyclase enzyme (e.g., altOAC) sequences comprise an amino acid sequence that is at least 50% identical to the sequence set forth in either SEQ ID NO: 385 or SEQ ID NO: 386. Not all proteins that contain a DABB domain will be capable of catalyzing the OAC reaction, but the presence of a DABB domain is likely in sequences that are catalytically capable of catalyzing the OAC reaction. Similarly, PKSC enzymes are likely to contain structural interaction motifs to facilitate their recruitment to and interaction with other subunits in a multi protein complex.

PKSC enzymes were chosen to belong within (derived from) the following protein superfamilies: Abhydrolase superfamily, ABM superfamily, Acyl_transf_1 superfamily, AdoMet_MTases superfamily, AFD_class_I superfamily, BioC superfamily, cond_enzymes superfamily, cupin_RmlC-like superfamily, entF superfamily, EthD superfamily, fabG superfamily, hot_dog superfamily, MDR superfamily, NADB_Rossmann superfamily, NRPS_MxcG superfamily, omega_3_PfaA superfamily, PKS_MbtD superfamily, PKS_NbtC superfamily, PP-binding superfamily, PRK12467 superfamily, PKS_MbtD superfamily, PKS_NbtC superfamily, PP-binding superfamily, and/or PRK12467 superfamily. These superfamilies have the corresponding accession numbers as provided by the National Center for Biotechnology Information (NCBI) structure database (NCBI, Conserved Protein Domain Families): c117068, c109936, c108282, c116912, c117173, c127680, c117068, c137044, c140423, c110022, c121494, c135902, c135333, c109938, c121454, c140423, c141646, c137173, c100509, c141573, c141574, c136129.

PKSC enzymes within these superfamilies of proteins were also chosen to include one or more of the following protein domains: A_NRPS, ACP, AcpP, acyl_carrier, Acyl_transf_1, ADH_zinc_N, AdoMet_MTases, alpha_am_amid, AMP-binding, BioC, CaiC, Cupin_2, cupin_RmlC-like, Dabb, enoyl_red, EntF, entF, FabD, fabG, ketoacyl-synt, KR, KR_2_FAS_SDR_x, KR_FAS_SDR_x, ManC, Methyltransf_11, Methyltransf_12, NRPS_MxcG, omega_3_PfaA, PKS, PKS_AT, PKS_DH, PKS_ER, PKS_KR, PKS_KS, PKS_MbtD, PKS_MbtD superfamily, PKS_NbtC, PKS_NbtC superfamily, PKS_PP, PKS_TE, PksD, PLN02752, PLN02836, PP-binding, PP-binding superfamily, PRK06333, PRK07314, PRK12467, PRK12467 superfamily, PS-DH, PT_fungal_PKS, PTZ00050, PTZ00354, Qor, quinone_pig3, SAT, Thioesterase, UbiE, and/or ubiE.

### Organisms Originating the Enzymes

The altOAC and PKSC enzymes can be naturally occurring enzymes, or enzymes derived from a naturally occurring enzyme, now known or later discovered, that occurs in any living organism, for example a bacterium, an archaeon, a protist, a fungus, an algae, an animal or a plant.

Many enzymes catalyze reactions of these classes using similar substrates, but have never been tested for activity on cannabinoids. To determine a source of an altOAC or PKSC enzyme, microbes can be screened for bioconversion activity of appropriate cannabinoids, after the methods of Abbott (1977). Enzymes from the above listed enzyme classes can be found, e.g., from the sequenced genomes, by cloning enzymes homologous to other cyclases, or by other cloning methods, thereby identifying good candidates for OAC activity. Organisms that make molecules similar to desired cannabinoids can also be identified from literature and those genomes searched as well to identify additional candidate enzymes. Bioinformatics methods to do this are provided in U.S. Pat. No. 10,671,632.

In some embodiments, the gene for the altOAC or PKSC enzyme is derived from a bacterium. It is envisioned that an altOAC or PKSC enzyme derived from any bacterium now known or later discovered can be utilized in the present invention. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order Bacteroidales, Marinilabiliales, Chitinophagales, Cytophagales, Flavobacteriales, Saprospirales, or Sphingopacteriales; phylum Balneolaeota, Caldiserica, Calditrichaeota, or Chlamydiae, including class Balneolia, Caldisericia, Calditrichae, or Chlamydia, including order Balneolales, Caldisericales, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Ktedonobacterales, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales; phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillales, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, Iodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalassiales, Rickettsiales, Sneathiellales, Sphingomonadales, Burkholderiales, Ferritrophicales, Ferrovales, Neisseriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergi stales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the gene for the enzyme is derived from an archaeon. It is envisioned that an altOAC or PKSC enzyme derived from any archaeon now known or later discovered can be utilized in the present invention. For example, the archaeon can be from phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomassiliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylium Odinarchaeota, Thorarchaeota, Lokiarchaeota, Helarchaeota, or Heimdallarchaeota.

In additional embodiments, the gene for the altOAC or PKSC enzyme is derived from a fungus. It is envisioned that an altOAC or PKSC enzyme from any fungus now known or later discovered can be utilized in the present invention. This includes but is not limited to the phyla Chytridiomycota, Basidiomycota, Ascomycota, Blastocladiomycota, Ascomycota, Microsporidia, Basidiomycota, Glomeromycota, Symbiomycota, and Neocallimastigomycota. For example, the fungus can be from the phylum Ascomycota, including classes and orders Pezizomycotina, Arthoniomycetes, Coniocybomycetes, Dothideomycetes, Eurotiomycetes, Geoglossomycetes, Laboulbeniomycetes, Lecanoromycetes, Leotiomycetes, Lichinomycetes, Orbiliomycetes, Pezizomycetes, Sordariomycetes, Xylonomycetes, Lahmiales, Itchiclahmadion, Triblidiales, Saccharomycotina, Saccharomycetes, Taphrinomycotina, Archaeorhizomyces, Neolectomycetes, Pneumocystidomycetes, Schizosaccharomycetes, Taphrinomycetes; phylum Basidiomycota including subphyla or classes Pucciniomycotina, Ustilaginomycotina, Wallemiomycetes, and Entorrhizomycetes; subphylum Agaricomycotina including classes Tremellomycetes, Dacrymycetes, and Agaricomycetes; phylum Symbiomycota, including class Entorrhizomycota; subphylum Ustilaginomycotina including classes Ustilaginomycetes and Exobasidiomycetes; phylum Glomeromycota including classes Archaeosporomycetes, Glomeromycetes, and Paraglomeromycetes; subphylum Pucciniomycotina including orders and classes: Pucciniomycotina, Cystobasidiomycetes, Agaricostilbomycetes, Microbotryomycetes, Atractiellomycetes, Classiculomycetes, Mixiomycetes, and Cryptomycocolacomycetes; subphylum incertae sedis Mucoromyceta including orders Calcarisporiellomycota and Mucoromycota; phylum Mortierellomyceta including class Mortierellomycota; subphylum incertae sedis Entomophthoromycotina including order Entomophthorales; phylum Zoopagomyceta including classes Basidiobolomycota, Entomophthoromycota, Kickxellomycota, and Zoopagomycotina; subphylum incertae sedis Mucoromycotina including orders Mucorales, Endogonales, and Mortierellales; phylum Neocallimastigomycota including class Neocallimastigomycetes; phylum Blastocladiomycota including classes Physodermatomycetes and Blastocladiomycetes; phylum Rozellomyceta including classes Rozellomycota and Microsporidia; phylum Aphelidiomyceta including class Aphelidiomycota; Chytridiomyceta including classes Chytridiomycetes and Monoblepharidomycetes; and phylum Oomycota including classes or orders Leptomitales, Myzocytiopsidales, Olpidiopsidales, Peronosporales, Pythiales, Rhipidiales, Salilagenidiales, Saprolegniales, Sclerosporales, Anisolpidiales, Lagenismatales, Rozellopsidales, and Haptoglossales.

### Nucleic Acids

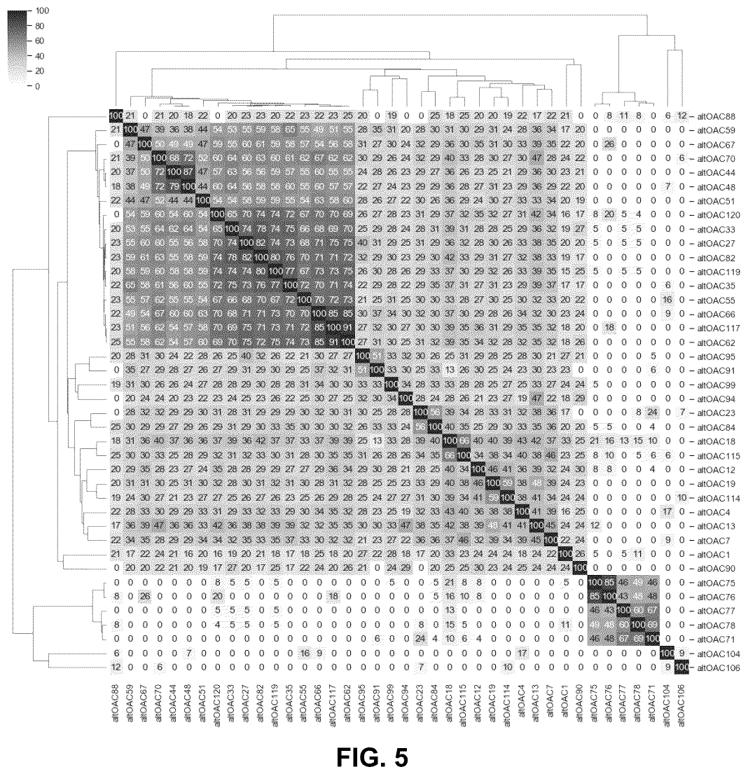

The present invention is additionally directed to nucleic acids encoding any of the above-described altOAC and PKSC enzymes including but are not limited to the microbial and non-Cannabis plant OACs. Gene sequences can be determined using the techniques disclosed in U.S. Pat. No. 10,671,632, or by any other method known in the art. Table 1 provides SEQ ID NOs for nucleic acid and amino acid sequences listed in the sequence listing provided below. A Clustermap showing homologies between the amino acid sequences of select altOACs is provided in FIG. 5.

TABLE 1

| | Codon Optimized | Amino Acid Sequence |
|---|---|---|
| NAME | Nucleic Acid Sequence | for Isolated Protein |
| PKSC1A | SEQ ID NO: 1 | SEQ ID NO: 193 |
| PKSC1B | SEQ ID NO: 2 | SEQ ID NO: 194 |
| PKSC1C | SEQ ID NO: 3 | SEQ ID NO: 195 |
| PKSC2A | SEQ ID NO: 4 | SEQ ID NO: 196 |
| PKSC2B | SEQ ID NO: 5 | SEQ ID NO: 197 |
| PKSC2C | SEQ ID NO: 6 | SEQ ID NO: 198 |
| PKSC3A | SEQ ID NO: 7 | SEQ ID NO: 199 |
| PKSC3B | SEQ ID NO: 8 | SEQ ID NO: 200 |
| PKSC3C | SEQ ID NO: 9 | SEQ ID NO: 201 |
| PKSC4A | SEQ ID NO: 10 | SEQ ID NO: 202 |
| PKSC4B | SEQ ID NO: 11 | SEQ ID NO: 203 |
| PKSC4C | SEQ ID NO: 12 | SEQ ID NO: 204 |
| PKSC5A | SEQ ID NO: 13 | SEQ ID NO: 205 |
| PKSC5B | SEQ ID NO: 14 | SEQ ID NO: 206 |
| PKSC5C | SEQ ID NO: 15 | SEQ ID NO: 207 |
| PKSC6A | SEQ ID NO: 16 | SEQ ID NO: 208 |
| PKSC6B | SEQ ID NO: 17 | SEQ ID NO: 209 |
| PKSC6C | SEQ ID NO: 18 | SEQ ID NO: 210 |
| PKSC7A | SEQ ID NO: 19 | SEQ ID NO: 211 |
| PKSC7B | SEQ ID NO: 20 | SEQ ID NO: 212 |
| PKSC7C | SEQ ID NO: 21 | SEQ ID NO: 213 |
| PKSC8A | SEQ ID NO: 22 | SEQ ID NO: 214 |
| PKSC8B | SEQ ID NO: 23 | SEQ ID NO: 215 |
| PKSC8C | SEQ ID NO: 24 | SEQ ID NO: 216 |
| PKSC9A | SEQ ID NO: 25 | SEQ ID NO: 217 |
| PKSC9B | SEQ ID NO: 26 | SEQ ID NO: 218 |
| PKSC9C | SEQ ID NO: 27 | SEQ ID NO: 219 |
| PKSC10A | SEQ ID NO: 28 | SEQ ID NO: 220 |
| PKSC10B | SEQ ID NO: 29 | SEQ ID NO: 221 |
| PKSC10C | SEQ ID NO: 30 | SEQ ID NO: 222 |
| PKSC11A | SEQ ID NO: 31 | SEQ ID NO: 223 |
| PKSC11B | SEQ ID NO: 32 | SEQ ID NO: 224 |
| PKSC11C | SEQ ID NO: 33 | SEQ ID NO: 225 |
| PKSC12A | SEQ ID NO: 34 | SEQ ID NO: 226 |
| PKSC12B | SEQ ID NO: 35 | SEQ ID NO: 227 |
| PKSC12C | SEQ ID NO: 36 | SEQ ID NO: 228 |
| PKSC13A | SEQ ID NO: 37 | SEQ ID NO: 229 |
| PKSC13B | SEQ ID NO: 38 | SEQ ID NO: 230 |
| PKSC13C | SEQ ID NO: 39 | SEQ ID NO: 231 |
| PKSC14A | SEQ ID NO: 40 | SEQ ID NO: 232 |
| PKSC14B | SEQ ID NO: 41 | SEQ ID NO: 233 |
| PKSC14C | SEQ ID NO: 42 | SEQ ID NO: 234 |
| PKSC15A | SEQ ID NO: 43 | SEQ ID NO: 235 |
| PKSC15B | SEQ ID NO: 44 | SEQ ID NO: 236 |
| PKSC15C | SEQ ID NO: 45 | SEQ ID NO: 237 |
| PKSC16A | SEQ ID NO: 46 | SEQ ID NO: 238 |
| PKSC16B | SEQ ID NO: 47 | SEQ ID NO: 239 |
| PKSC16C | SEQ ID NO: 48 | SEQ ID NO: 240 |
| PKSC17A | SEQ ID NO: 49 | SEQ ID NO: 241 |
| PKSC17B | SEQ ID NO: 50 | SEQ ID NO: 242 |
| PKSC17C | SEQ ID NO: 51 | SEQ ID NO: 243 |
| PKSC18A | SEQ ID NO: 52 | SEQ ID NO: 244 |
| PKSC18B | SEQ ID NO: 53 | SEQ ID NO: 245 |
| PKSC18C | SEQ ID NO: 54 | SEQ ID NO: 246 |
| PKSC19A | SEQ ID NO: 55 | SEQ ID NO: 247 |
| PKSC19B | SEQ ID NO: 56 | SEQ ID NO: 248 |
| PKSC19C | SEQ ID NO: 57 | SEQ ID NO: 249 |
| PKSC20A | SEQ ID NO: 58 | SEQ ID NO: 250 |
| PKSC20B | SEQ ID NO: 59 | SEQ ID NO: 251 |
| PKSC20C | SEQ ID NO: 60 | SEQ ID NO: 252 |
| PKSC21C | SEQ ID NO: 61 | SEQ ID NO: 253 |
| PKSC22C | SEQ ID NO: 62 | SEQ ID NO: 254 |
| PKSC23C | SEQ ID NO: 63 | SEQ ID NO: 255 |
| PKSC24B | SEQ ID NO: 64 | SEQ ID NO: 256 |
| PKSC25B | SEQ ID NO: 65 | SEQ ID NO: 257 |
| PKSC26C | SEQ ID NO: 66 | SEQ ID NO: 258 |
| PKSC27C | SEQ ID NO: 67 | SEQ ID NO: 259 |
| altOAC1 | SEQ ID NO: 68 | SEQ ID NO: 260 |
| altOAC2 | SEQ ID NO: 69 | SEQ ID NO: 261 |
| altOAC3 | SEQ ID NO: 70 | SEQ ID NO: 262 |
| altOAC4 | SEQ ID NO: 71 | SEQ ID NO: 263 |
| altOAC5 | SEQ ID NO: 72 | SEQ ID NO: 264 |
| altOAC6 | SEQ ID NO: 73 | SEQ ID NO: 265 |
| altOAC7 | SEQ ID NO: 74 | SEQ ID NO: 266 |
| altOAC8 | SEQ ID NO: 75 | SEQ ID NO: 267 |
| altOAC9 | SEQ ID NO: 76 | SEQ ID NO: 268 |
| altOAC10 | SEQ ID NO: 77 | SEQ ID NO: 269 |
| altOAC11 | SEQ ID NO: 78 | SEQ ID NO: 270 |
| altOAC12 | SEQ ID NO: 79 | SEQ ID NO: 271 |
| altOAC13 | SEQ ID NO: 80 | SEQ ID NO: 272 |
| altOAC14 | SEQ ID NO: 81 | SEQ ID NO: 273 |
| altOAC15 | SEQ ID NO: 82 | SEQ ID NO: 274 |
| altOAC16 | SEQ ID NO: 83 | SEQ ID NO: 275 |
| altOAC17 | SEQ ID NO: 84 | SEQ ID NO: 276 |
| altOAC18 | SEQ ID NO: 85 | SEQ ID NO: 277 |
| altOAC19 | SEQ ID NO: 86 | SEQ ID NO: 278 |
| altOAC20 | SEQ ID NO: 87 | SEQ ID NO: 279 |
| altOAC21 | SEQ ID NO: 88 | SEQ ID NO: 280 |
| altOAC22 | SEQ ID NO: 89 | SEQ ID NO: 281 |
| altOAC23 | SEQ ID NO: 90 | SEQ ID NO: 282 |
| altOAC24 | SEQ ID NO: 91 | SEQ ID NO: 283 |
| altOAC25 | SEQ ID NO: 92 | SEQ ID NO: 284 |
| altOAC26 | SEQ ID NO: 93 | SEQ ID NO: 285 |
| altOAC27 | SEQ ID NO: 94 | SEQ ID NO: 286 |
| altOAC28 | SEQ ID NO: 95 | SEQ ID NO: 287 |
| altOAC29 | SEQ ID NO: 96 | SEQ ID NO: 288 |
| altOAC30 | SEQ ID NO: 97 | SEQ ID NO: 289 |
| altOAC31 | SEQ ID NO: 98 | SEQ ID NO: 290 |
| altOAC32 | SEQ ID NO: 99 | SEQ ID NO: 291 |
| altOAC33 | SEQ ID NO: 100 | SEQ ID NO: 292 |
| altOAC34 | SEQ ID NO: 101 | SEQ ID NO: 293 |
| altOAC35 | SEQ ID NO: 102 | SEQ ID NO: 294 |
| altOAC36 | SEQ ID NO: 103 | SEQ ID NO: 295 |
| altOAC37 | SEQ ID NO: 104 | SEQ ID NO: 296 |
| altOAC38 | SEQ ID NO: 105 | SEQ ID NO: 297 |
| altOAC39 | SEQ ID NO: 106 | SEQ ID NO: 298 |
| altOAC40 | SEQ ID NO: 107 | SEQ ID NO: 299 |
| altOAC41 | SEQ ID NO: 108 | SEQ ID NO: 300 |
| altOAC42 | SEQ ID NO: 109 | SEQ ID NO: 301 |
| altOAC43 | SEQ ID NO: 110 | SEQ ID NO: 302 |
| altOAC44 | SEQ ID NO: 111 | SEQ ID NO: 303 |
| altOAC45 | SEQ ID NO: 112 | SEQ ID NO: 304 |
| altOAC46 | SEQ ID NO: 113 | SEQ ID NO: 305 |
| altOAC47 | SEQ ID NO: 114 | SEQ ID NO: 306 |
| altOAC48 | SEQ ID NO: 115 | SEQ ID NO: 307 |
| altOAC49 | SEQ ID NO: 116 | SEQ ID NO: 308 |
| altOAC50 | SEQ ID NO: 117 | SEQ ID NO: 309 |
| altOAC51 | SEQ ID NO: 118 | SEQ ID NO: 310 |
| altOAC52 | SEQ ID NO: 119 | SEQ ID NO: 311 |
| altOAC53 | SEQ ID NO: 120 | SEQ ID NO: 312 |
| altOAC54 | SEQ ID NO: 121 | SEQ ID NO: 313 |
| altOAC55 | SEQ ID NO: 122 | SEQ ID NO: 314 |
| altOAC56 | SEQ ID NO: 123 | SEQ ID NO: 315 |
| altOAC57 | SEQ ID NO: 124 | SEQ ID NO: 316 |
| altOAC58 | SEQ ID NO: 125 | SEQ ID NO: 317 |
| altOAC59 | SEQ ID NO: 126 | SEQ ID NO: 318 |
| altOAC60 | SEQ ID NO: 127 | SEQ ID NO: 319 |
| altOAC61 | SEQ ID NO: 128 | SEQ ID NO: 320 |
| altOAC62 | SEQ ID NO: 129 | SEQ ID NO: 321 |
| altOAC63 | SEQ ID NO: 130 | SEQ ID NO: 322 |
| altOAC64 | SEQ ID NO: 131 | SEQ ID NO: 323 |
| altOAC65 | SEQ ID NO: 132 | SEQ ID NO: 324 |
| altOAC66 | SEQ ID NO: 133 | SEQ ID NO: 325 |
| altOAC67 | SEQ ID NO: 134 | SEQ ID NO: 326 |
| altOAC68 | SEQ ID NO: 135 | SEQ ID NO: 327 |
| altOAC69 | SEQ ID NO: 136 | SEQ ID NO: 328 |
| altOAC70 | SEQ ID NO: 137 | SEQ ID NO: 329 |
| altOAC71 | SEQ ID NO: 138 | SEQ ID NO: 330 |
| altOAC72 | SEQ ID NO: 139 | SEQ ID NO: 331 |
| altOAC73 | SEQ ID NO: 140 | SEQ ID NO: 332 |
| altOAC74 | SEQ ID NO: 141 | SEQ ID NO: 333 |
| altOAC75 | SEQ ID NO: 142 | SEQ ID NO: 334 |
| altOAC76 | SEQ ID NO: 143 | SEQ ID NO: 335 |
| altOAC77 | SEQ ID NO: 144 | SEQ ID NO: 336 |
| altOAC78 | SEQ ID NO: 145 | SEQ ID NO: 337 |
| altOAC79 | SEQ ID NO: 146 | SEQ ID NO: 338 |
| altOAC80 | SEQ ID NO: 147 | SEQ ID NO: 339 |
| altOAC81 | SEQ ID NO: 148 | SEQ ID NO: 340 |
| altOAC82 | SEQ ID NO: 149 | SEQ ID NO: 341 |
| altOAC83 | SEQ ID NO: 150 | SEQ ID NO: 342 |
| altOAC84 | SEQ ID NO: 151 | SEQ ID NO: 343 |
| altOAC85 | SEQ ID NO: 152 | SEQ ID NO: 344 |
| altOAC86 | SEQ ID NO: 153 | SEQ ID NO: 345 |
| altOAC87 | SEQ ID NO: 154 | SEQ ID NO: 346 |
| altOAC88 | SEQ ID NO: 155 | SEQ ID NO: 347 |
| altOAC89 | SEQ ID NO: 156 | SEQ ID NO: 348 |
| altOAC90 | SEQ ID NO: 157 | SEQ ID NO: 349 |
| altOAC91 | SEQ ID NO: 158 | SEQ ID NO: 350 |
| altOAC92 | SEQ ID NO: 159 | SEQ ID NO: 351 |
| altOAC93 | SEQ ID NO: 160 | SEQ ID NO: 352 |
| altOAC94 | SEQ ID NO: 161 | SEQ ID NO: 353 |
| altOAC95 | SEQ ID NO: 162 | SEQ ID NO: 354 |
| altOAC96 | SEQ ID NO: 163 | SEQ ID NO: 355 |
| altOAC97 | SEQ ID NO: 164 | SEQ ID NO: 356 |
| altOAC98 | SEQ ID NO: 165 | SEQ ID NO: 357 |
| altOAC99 | SEQ ID NO: 166 | SEQ ID NO: 358 |
| altOAC100 | SEQ ID NO: 167 | SEQ ID NO: 359 |
| altOAC101 | SEQ ID NO: 168 | SEQ ID NO: 360 |
| altOAC102 | SEQ ID NO: 169 | SEQ ID NO: 361 |
| altOAC103 | SEQ ID NO: 170 | SEQ ID NO: 362 |
| altOAC104 | SEQ ID NO: 171 | SEQ ID NO: 363 |
| altOAC105 | SEQ ID NO: 172 | SEQ ID NO: 364 |
| altOAC106 | SEQ ID NO: 173 | SEQ ID NO: 365 |
| altOAC107 | SEQ ID NO: 174 | SEQ ID NO: 366 |
| altOAC108 | SEQ ID NO: 175 | SEQ ID NO: 367 |
| altOAC109 | SEQ ID NO: 176 | SEQ ID NO: 368 |
| altOAC110 | SEQ ID NO: 177 | SEQ ID NO: 369 |
| altOAC111 | SEQ ID NO: 178 | SEQ ID NO: 370 |
| altOAC112 | SEQ ID NO: 179 | SEQ ID NO: 371 |
| altOAC113 | SEQ ID NO: 180 | SEQ ID NO: 372 |
| altOAC114 | SEQ ID NO: 181 | SEQ ID NO: 373 |
| altOAC115 | SEQ ID NO: 182 | SEQ ID NO: 374 |
| altOAC116 | SEQ ID NO: 183 | SEQ ID NO: 375 |
| altOAC117 | SEQ ID NO: 184 | SEQ ID NO: 376 |
| altOAC118 | SEQ ID NO: 185 | SEQ ID NO: 377 |
| altOAC119 | SEQ ID NO: 186 | SEQ ID NO: 378 |
| csOAC | SEQ ID NO: 187 | SEQ ID NO: 379 |

In some embodiments, the nucleic acids are codon optimized to improve expression, e.g., using techniques as disclosed in U.S. Pat. No. 10,435,727. More specifically, optimized nucleotide sequences are generated based on a number of considerations: (1) For each amino acid of the recombinant polypeptide to be expressed, a codon (triplet of nucleotide bases) is selected based on the frequency of each codon in the Saccharomyces cerevisiae genome; the codon can be chosen to be the most frequent codon or can be selected probabilistically based on the frequencies of all possible codons. (2) In order to prevent DNA cleavage due to a restriction enzyme, certain restriction sites are removed by changing codons that cover those sites. (3) To prevent low-complexity regions, long repeats (sequences of any single base longer than five bases) are modified. (2) and (3) are performed recursively to ensure that codon modification does not lead to additional undesirable sequences. (4) A ribosome binding site is added to the N-terminus. (5) A stop codon is added.

In various embodiments, the nucleic acids further comprise additional nucleic acids encoding amino acids that are not part of the altOAC or PKSC enzyme. In some of these embodiments, the additional sequences encode additional amino acids present when the nucleic acid is translated, encoding, for example, an additional protein domain, with or without a linker sequence, creating a fusion protein. Other examples are localization sequences, i.e., signals directing the localization of the folded protein to a specific subcellular compartment or membrane.

In some embodiments, the nucleic acids have, at the 5′ end, a nucleic acid encoding codon optimized cofolding peptides to create a fusion protein, e.g., comprising SEQ ID NOs:188-192 (Table 2), joining the sequences together to form a fusion polypeptide, e.g., comprising the amino acid sequence of SEQ ID NO:380-384 fused at the N terminus of the enzyme polypeptide, generating recombinant fusion polypeptides.

TABLE 2

| | Codon Optimized | Amino Acid Sequence |
|---|---|---|
| NAME | Nucleic Acid Sequence | for Isolated Protein |
| MBP | SEQ ID NO: 188 | SEQ ID NO: 380 |
| VEN | SEQ ID NO: 189 | SEQ ID NO: 381 |
| MST | SEQ ID NO: 190 | SEQ ID NO: 382 |
| OSP | SEQ ID NO: 191 | SEQ ID NO: 383 |
| OLE | SEQ ID NO: 192 | SEQ ID NO: 384 |

Further provided are non-naturally occurring nucleic acids that encode an enzyme having the enzymatic activity of any of the non-naturally occurring altOAC or PKSC enzymes described above, or a naturally occurring enzyme having OAC activity. The nucleic acids may be codon optimized, e.g., for production in yeast.

In some embodiments, the nucleic acid comprises additional nucleotide sequences that are not translated. Nonlimiting examples include promoters, terminators, barcodes, Kozak sequences, targeting sequences, and enhancer elements. Particularly useful here are promoters that are functional in yeast.

Expression of a gene encoding an enzyme is determined by the promoter controlling the gene. In order for a gene to be expressed, a promoter must be present within 1,000 nucleotides upstream of the gene. A gene is generally cloned under the control of a desired promoter. The promoter regulates the amount of enzyme expressed in the cell and also the timing of expression, or expression in response to external factors such as sugar source.

Any promoter now known or later discovered can be utilized to drive the expression of the altOAC or PKSC genes described herein. See e.g. http://parts.igem.org/Yeast for a listing of various yeast promoters. Exemplary promoters listed in Table 3 below drive strong expression, constant gene expression, medium or weak gene expression, or inducible gene expression. Inducible or repressible gene expression is dependent on the presence or absence of a certain molecule. For example, the GAL1, GAL7, and GAL10 promoters are activated by the presence of the sugar galactose and repressed by the presence of the sugar glucose. The HO promoter is active and drives gene expression only in the presence of the alpha factor peptide. The HXT1 promoter is activated by the presence of glucose while the ADH2 promoter is repressed by the presence of glucose.

TABLE 3
Exemplary yeast promoters

| Strong | Medium and weak | Inducible/repressible |
|---|---|---|
| constitutive promoters | constitutive promoters | promoters |
| TEF1 | STE2 | GAL1 |
| PGK1 | TPI1 | GAL7 |
| PGI1 | PYK1 | GAL10 |
| TDH3 | | HO |
| | | HXT1 |
| | | ADH2 |

In various embodiments, the nucleic acid is in an expression cassette, e.g., a yeast expression cassette. Any yeast expression cassette capable of expressing the enzyme in a yeast cell can be utilized. In some embodiments, the expression cassette consists of a nucleic acid encoding an altOAC or PKSC with a promoter.

Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. See e.g. http://parts.igem.org/Yeast for a listing of various yeast vectors.

A nonlimiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter (see Table 3). Other exemplary vectors include pRS series plasmids.

### Host Cells

The present invention is also directed to genetically engineered host cells that comprise the above-described nucleic acids. Such cells may be, e.g., any species of filamentous fungus, including but not limited to any species of Aspergillus, which have been genetically altered to produce precursor molecules, intermediate molecules, or cannabinoid molecules. Host cells may also be any species of bacteria, including but not limited to Escherichia, Corynebacterium, Caulobacter, Pseudomonas, Streptomyces, Bacillus, or Lactobacillus.

In some embodiments, the genetically engineered host cell is a yeast cell, which may comprise any of the above-described expression cassettes, and capable of expressing the recombinant altOAC or PKSC enzyme encoded therein.

Any yeast cell capable of being genetically engineered can be utilized in these embodiments. Nonlimiting examples of such yeast cells include species of Saccharomyces, Candida, Pichia, Schizosaccharomyces, Scheffersomyces, Blakeslea, Rhodotorula, or Yarrowia.

These cells can achieve gene expression controlled by inducible promoter systems; natural or induced mutagenesis, recombination, and/or shuffling of genes, pathways, and whole cells performed sequentially or in cycles; overexpression and/or deletion of single or multiple genes and reducing or eliminating parasitic side pathways that reduce precursor concentration.

The host cells of the recombinant organism may also be engineered to produce any or all precursor molecules necessary for the biosynthesis of cannabinoids, including but not limited to olivetol (OL), farnesyl diphosphate (FPP) and geranyl diphosphate (GPP), hexanoic acid and hexanoyl-CoA, malonic acid and malonyl-CoA, dimethylallylpyrophosphate (DMAPP) and isopentenylpyrophosphate (IPP) as disclosed in U.S. Pat. No. 10,435,727.

The gene encoding the enzyme can be cloned into vectors with the proper regulatory elements for gene expression (e.g. promoter, terminator) and the derived plasmid can be confirmed by DNA sequencing. As an alternative to expression from an episomal plasmid, the gene encoding the enzyme may be inserted into the recombinant host genome. Integration may be achieved by a single or double cross-over insertion event of a plasmid, or by nuclease-based genome editing methods, as are known in the art e.g. CRISPR, TALEN and ZFR. Strains with the integrated gene can be screened by rescue of auxotrophy and genome sequencing. See, e.g., Green and Sambrook (2012).

To produce the desired cannabinoid, each candidate polypeptide may be introduced into a host cell genetically modified to contain all necessary components for cannabinoid biosynthesis using standard yeast cell transformation techniques (Green and Sambrook, 2012), e.g., other enzymes in the cannabinoid biosynthetic pathway such as PKS, geranyl pyrophosphate synthase (see, e.g., U.S. Provisional Patent Application 63/141,486), prenyltransferase (see, e.g., U.S. Provisional Patent Application 63/053,539), and the enzymes described in U.S. Provisional Patent Application 63/164,126. Cells are subjected to fermentation under conditions that activate the promoter controlling the candidate polypeptide (see, e.g., Table 2). The broth may be subsequently subjected to HPLC analysis.

In some embodiments, for recombinant enzyme purification, the gene encoding the enzyme is cloned into an expression vector such as the pET expression vectors from Novagen, transformed into a protease deficient strain of E. coli such as BL21 and expressed by induction with IPTG. The protein of interest may be tagged with a common tag to facilitate purification, e.g. hexahistidine, GST, calmodulin, TAP, AP, CAT, HA, FLAG, MBP etc. Coexpression of a bacterial chaperone such as dnaK, GroES/GroEL or SecY may help facilitate protein folding. See Green and Sambrook (2012).

### Methods

The present invention is also directed to a method of converting a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃,

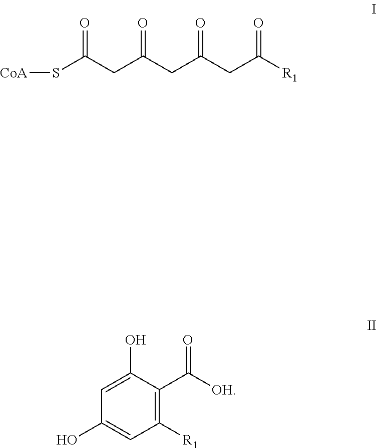

The method comprises contacting the polyketide with any of the olivetolic acid cyclase (OAC) enzymes described herein in a manner and for a time sufficient to convert the polyketide (I) into the resorcyclic acid derivative (II).

In some embodiments, the method is carried out in vitro (outside of a cell). In other embodiments, the polyketide and the OAC enzyme are present in a living microorganism, for example any of the recombinant host cells described above, such as a yeast cell. In various embodiments, the recombinant host cell can further convert the resorcyclic acid derivative into a cannabinoid, and/or synthesize the polyketide from precursors.

In various specific embodiments, samples from fermentations of recombinant hosts expressing the cannabinoid pathway with fungal olivetolic acid cyclases outlined above are: (i) prepared and extracted using a combination of fermentation, dissolution, and purification steps; and (ii) analyzed by HPLC for the presence of directing molecules, precursor molecules, intermediate molecules, and target molecules such as OA, OL and common variants.

In various embodiments, the host cells are provided with various feedstocks to drive production of the desired ergolines, e.g., glucose, fructose, sucrose, galactose, raffinose, maltose, ethanol, xylose, fatty acids, glycerol, acetate, molasses, malt syrup, corn steep liquor, dairy, flour, protein powder, olive mill waste, fish waste, etc. for example as discussed in U.S. patent application Ser. No. 17/068,636.

In various embodiments, an inducer is used to activate the expression of the OAC pathway, such as the expression of altOAC or PKSC, or a combination of PKSC genes, or a combination of the cannabis OAC, csOAC, with altOAC and PKSC genes. Inducers include: galactose, glycerol, sucrose, maltose, lactose, glucose, hexanoic acid, hexanol, butyric acid, butanol, tributyrin, xylose, copper, and/or zinc.

In some embodiments, a vitamin mixture is added to a fermentation. Such as mixture can contain: choline chloride, niacin, pyridoxine hydrochloride, riboflavin, calcium pantothenate, para-aminobenzoic acid (PABA), thiamine HCL, biotin, cyanocobalamin, and/or folic acid, and mineral mixes, which can include calcium chloride dihydrate, ferrous sulfate heptahydrate, manganese (II) sulfate monohydrate, copper sulfate pentahydrate, zinc sulfate heptahydrate, magnesium chloride, and solutes, such as glycerol, up to 10% v/v. Since these are oxidoreductase reactions, they may be stimulated by changing the redox potential of the culture. This can be accomplished by addition of oxidants such as H₂O₂, sulfuric acid (H2SO4), nitric acid (HNO3), potassium permanganate (KMnO4), and/or Fenton's reagent, antioxidants such as ascorbic acid, butylated hydroxyanisole (BHA), and/or butylated hydroxytoluene (BHT), or reductants such as 2-Mercaptoethanol (B-ME), dithiothreitol (DTT), glutathione, cysteine hydrochloride, and/or tris(2-carboxyethyl)phosphine (TCEP)

The following enumerated embodiments are representative of the invention:

1. An olivetolic acid cyclase (OAC) enzyme derived from a microorganism or a non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃, wherein (I) and (II) have the following structural formulas:

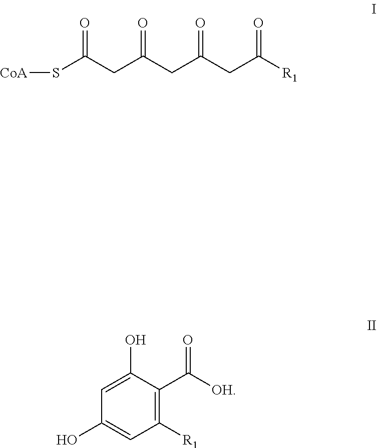

2. The enzyme of embodiment 1, wherein the microorganism is a bacterium or a fungus.

3. The enzyme of embodiment 1, comprising one or more mutations that increase specificity for particular R₁ alkyl chain length.

4. The enzyme of any one of embodiments 1-3, having an amino acid sequence that has less than 50% homology to the sequence set forth in SEQ ID NO: 379.

5. The enzyme of any one of embodiments 1-3, having an amino acid sequence that that is at least 95% identical to any one of the sequences set forth in SEQ ID NO: 193-SEQ ID NO: 378.

6. The enzyme of any one of embodiments 1-3, having an amino acid sequence that that is at least 50% identical to the sequence set forth in either SEQ ID NO: 385 or SEQ ID NO: 386.

7. The enzyme of any one of embodiments 1-3, having an amino acid sequence that that is at least 20% identical to the sequence set forth in SEQ ID NO: 334.

8. The enzyme of any one of embodiments 1-3, having an amino acid sequence that that is at least 50% identical to any one of the sequences set forth in SEQ ID NO: 193-SEQ ID NO: 195.

9. The enzyme of any one of embodiments 1-3, wherein the catalysis of the polyketide cyclization is by a mechanism selected from the group consisting of C2-C7 aldol condensation, Diekmann condensation, Claisen condensation, and Knoevenagel condensation.

10. An isolated nucleic acid encoding the enzyme of any one of embodiments 1-9.

11. The isolated nucleic acid of embodiment 10, which is codon optimized for production in yeast.

12. The codon-optimized nucleic acid of embodiment 11, inserted in a vector configured for replication and protein expression in yeast cells.

13. The isolated nucleic acid of any one of embodiments 10-12, having a nucleic acid sequence that is at least 95% identical to a sequence selected from the group consisting of SEQ ID NO: 1-SEQ ID NO: 186.

14. An expression cassette comprising the isolated nucleic acid of any one of embodiments 10-13.

15. The expression cassette of embodiment 14, which is a yeast expression cassette.

16. The expression cassette of embodiment 14 or embodiment 15, further comprising a nucleic acid fragment at the 5′ end of the isolated nucleic acid, wherein the nucleic acid fragment encodes a codon optimized cofolding peptide.

17. The expression cassette of embodiment 16, wherein the codon optimized cofolding peptide has an amino acid sequence that is at least 95% identical to a sequence selected from the group consisting of SEQ ID NO: 380-384.

18. A recombinant microorganism comprising the expression cassette of any one of embodiments 14-17, that expresses the engineered OAC enzyme encoded therein.

19. The recombinant microorganism of embodiment 18, which is a yeast cell.

20. The yeast cell of embodiment 19, which is a species of Saccharomyces, Candida, Pichia, Schizosaccharomyces, Scheffersomyces, Blakeslea, Rhodotorula, Aspergillus or Yarrowia.

21. The recombinant microorganism of any one of embodiments 18-20, further expressing at least one other enzyme in a cannabinoid biosynthetic pathway.

22. The recombinant microorganism of embodiment 21, wherein the at least one other enzyme is an OAC enzyme having an amino acid sequence that is at least 80% identical to the sequence set forth in SEQ ID NO: 379.

23. The recombinant microorganism of embodiment 21, wherein the at least one other enzyme is a polyketide synthase or a prenyltransferase.

24. The recombinant microorganism of any one of embodiments 21-23, capable of synthesizing a cannabinoid.

25. The recombinant microorganism of any one of embodiments 21-24, which is a yeast cell.

26. A method of converting a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃, wherein (I) and (II) have the following structural formulas:

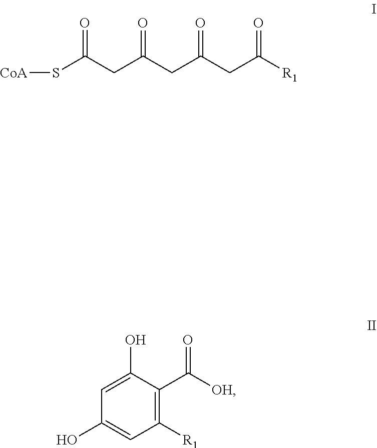

and wherein the method comprises contacting the polyketide with the olivetolic acid cyclase (OAC) enzyme of any one of embodiments 1-10 in a manner and for a time sufficient to convert the polyketide (I) into the resorcyclic acid derivative (II).

In preferred embodiments of the method, the olivetolic acid cyclase (OAC) enzyme catalyzes cyclization of the polyketide by a mechanism selected from the group consisting of C2-C7 aldol condensation, Diekmann condensation, Claisen condensation, and Knoevenagel condensation.

27. The method of embodiment 26, wherein the polyketide and the OAC enzyme are in vitro.

28. The method of embodiment 26, wherein the polyketide and the OAC enzyme are present in a living microorganism.

29. The method of embodiment 28, wherein the living microorganism is the recombinant microorganism of any one of embodiments 18-25.

30. The method of embodiment 29, wherein the living microorganism is the yeast cell of embodiment 25, and wherein the resorcyclic acid derivative is converted into a cannabinoid in the yeast cell.

### REFERENCES

Abbott et al., 1977, Experientia 33:718-720.
Carvalho et al., 2017, FEMS Yeast Res. 17:fox037.
Committee on the Health Effects of Marijuana, 2017, The Health Effects of Cannabis and Cannabinoids: The Current State of Evidence and Recommendations for Research, National Academies Press.
Gagne et al., 2012, Proc. Natl. Acad. Sci. USA 109:12811-12816.
Green and Sambrook, 2012, Molecular Cloning: A Laboratory Manual (Fourth Edition), Cold Spring Harbor Laboratory Press, Cold Spring Harbor, N.Y.
Gülck and Møller, 2020, Trends in Plant Science 25:985-1004.
Herbst et al., 2018, Nat. Prod. Rep. 35:1046.
Luo et al., 2019, Nature 567:123-126.
Okorafor, Ikechukwu C., Mengbin Chen, and Yi Tang. “High-Titer Production of Olivetolic Acid and Analogs in Engineered Fungal Host Using a Nonplant Biosynthetic Pathway.” ACS synthetic biology 10.9 (2021): 2159-2166
Shen, B., 2003, Current Opinion in Chemical Biology 7:285-295.
Yang et al., 2016, FEBS J. 283:1088-1106.
U.S. Pat. No. 10,435,727.
U.S. Pat. No. 10,671,632.
U.S. Pat. No. 9,765,308 B2
U.S. Pat. No. 11,028,417
U.S. Pat. No. 10,988,785
U.S. Pat. No. 11,041,002
U.S. Pat. No. 10,837,031
U.S. Pat. No. 11,293,038
U.S. patent application Ser. No. 17/068,636.
U.S. Provisional Patent Application 63/035,692.
U.S. Provisional Patent Application 63/053,539.
U.S. Provisional Patent Application 63/141,486.
U.S. Provisional Patent Application 63/164,126.
US Patent Application Publication 2020/0063170.
US Patent Application Publication 2020/0063171.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

### EXAMPLES

The following examples are offered to illustrate but not to limit the methods, compositions, and uses provided herein. Certain aspects of the present invention, including, but not limited to, methods for producing cannabinoid compounds in microorganisms, such as yeast cells, were disclosed in the earlier published patents and patent publications U.S. Pat. Nos. 10,435,727; 10,671,632; 9,765,308; 11,028,417; 10,988,785; 11,041,002; 10,837,031, 11,293,038; 2020/0063170; 2020/0063171, the contents of which are incorporated herein by reference in its entirety.

### Example 1. Method of Growth

Modified host cells that produce olivetolic acid (OA), and/or divarinic acid, and downstream cannabinoid compounds, such as the altOAC- and PKSC-expressing strains disclosed herein, express engineered altOAC or PKSC biosynthesis genes and enzymes, singly or in combination. Combining two or more altOAC- or PKSC-expressing genes in a microorganism can increase yields of production of OA, divarinic acid, and downstream cannabinoid compounds. More specifically, the OA-producing strain herein is grown in a minimal, complete culture media containing yeast nitrogen base, amino acids, vitamins, ammonium sulfate, and a carbon source of glucose and galactose. The recombinant host cells are grown in 24-well plates or shake flasks in a volume range of 2 mL to 100 mL of media starting from an inoculation density of OD600 nm=1. The strains herein can be harvested during a fermentation period ranging from 12 hours onward from the start of pathway enzyme induction.

### Example 2. Expression of altOAC and PKSC in a Modified Host Organism

Construction of the Saccharomyces OA and cannabinoid is carried out via expression of the altOAC or PKSC genes, singly, or co-expressed with genes which encode the downstream cannabinoid enzymes which can consume olivetolic acid or divarinic acid to produce cannabigerolic acid or cannabigerovarinic acid, as described in synthase in a GPP-production host as described in PCT/US21/42090 filed on Jul. 16, 2021, and in the U.S. Provisional Patent Application No. 63/553,539. AltOAC and PKSC genes encode the enzymes that synthesize olivetolic acid or divarinic acid which serve as a precursor for synthesis of valuable cannabinoids. In particular, they serve as a prenyl acceptor for a cannabinoid prenyltransferase, which combines the prenyl acceptor with a prenyl donor, such geranyl-pyrophsphate (GPP), farnesyl pyrophosphate (FPP), and/or geranylgeranyl-pyrophosphate (GGPP). Recombinant genes for producing prenyl donors can be co-expressed with altOAC and PKSC genes, alongside with cannabinoid enzymes, as described in PCT/US22/13857 and in the U.S. Provisional Patent Application No. 63/141,486. The optimized altOAC and pKASC genes described herein are synthesized using DNA synthesis techniques known in the art. The optimized genes can be cloned into vectors with the proper regulatory elements for gene expression (e.g. promoter, terminator) and the derived plasmid can be confirmed by DNA sequencing. As an alternative to expression from an episomal plasmid, the optimized altOAC and PKSC genes are inserted into the recombinant host genome. Integration is achieved by a single cross-over insertion event of the plasmid. Strains with the integrated gene can be screened by rescue of auxotrophy and genome sequencing.

### Example 3. Expression of Recombinant Multiple altOAC and/or PKSC Genes in a Modified Host Organism

Construction of Saccharomyces cerevisiae altOAC and/or PKSC production strains is carried out via expression of 1) an altOAC gene in combination with the cannabis OAC csOAC, 2) a PKSC1A-PKSC20A genes with a PKSC1B-PKSC25B gene with a PKSC1C-PKSC27C gene, 3) a PKSC1A-PKSC20A genes with a PKSC1B-PKSC25B gene, 4) a PKSC1A-PKSC20A genes with a PKSC1B-PKSC25B gene with a PKSC1C-PKSC27C gene with the recombinant cannabis OAC csOAC gene, 5) a PKSC1C-PKSC27C gene, or 6) the recombinant cannabis OAC, csOAC, coexpressed with a PKSC1C-PKSC27C gene. The optimized altOAC, csOAC, and PKSC genes are synthesized using DNA synthesis techniques known in the art. The optimized gene can be cloned into vectors with the proper regulatory elements for gene expression (e.g. promoter, terminator) and the derived plasmid can be confirmed by DNA sequencing. Plasmids can be constructed to contain multiple expression cassettes to encode multiple genes on a single plasmid by methods known to those skilled in the art. As an alternative to expression from an episomal plasmid, the optimized combination of genes is inserted into the recombinant host genome. Integration is achieved by a single cross-over insertion event of the plasmid. Strains with the integrated gene can be screened by rescue of auxotrophy and genome sequencing.

### Example 4. Detection of Isolated Product

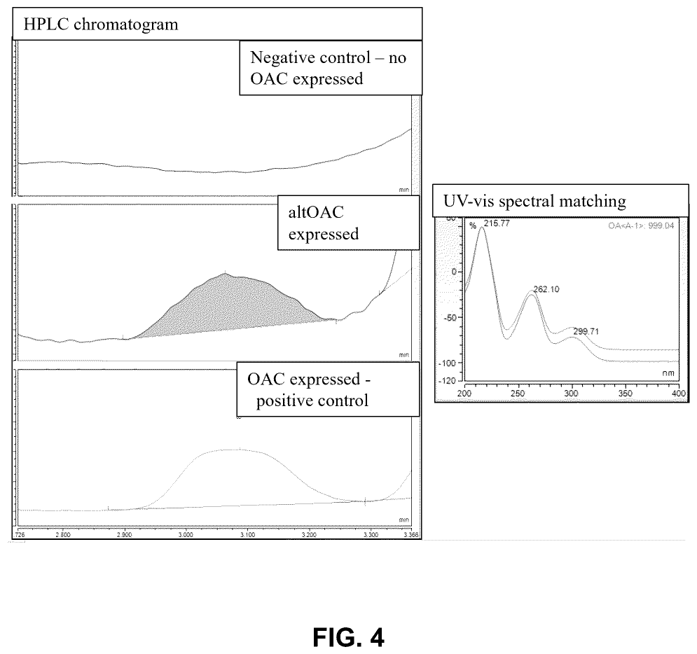

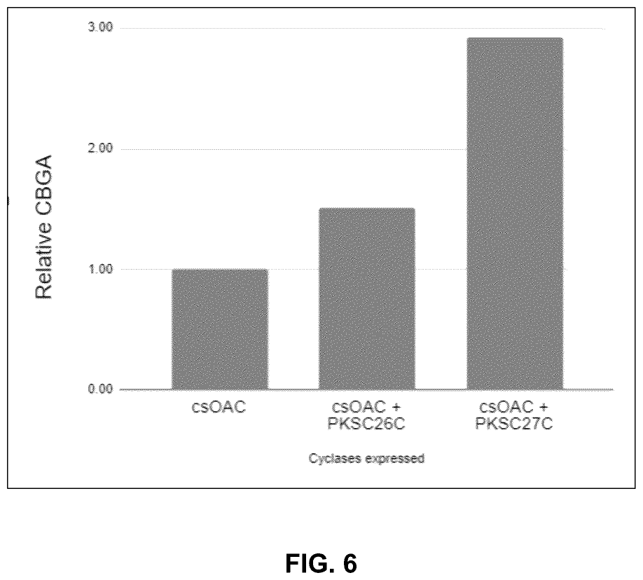

To identify fermentation-derived olivetolic acid, divarinic acid, olivetol, divarinol, their precursors, downstream cannabinoids, and all other products of a recombinant host expressing an engineered biosynthetic pathway for OA and cannabinoids, an Agilent 1100 series liquid chromatography (LC) system equipped with a reverse phase C18 column (Agilent Eclipse Plus C18, Santa Clara, CA, USA) is used. A gradient is used of mobile phase A (ultraviolet (UV) grade H2O+0.1% formic acid) and mobile phase B (UV grade acetonitrile+0.1% formic acid). Column temperature is set at 30° C. Compound absorbance is measured at 210 nm and 305 nm using a diode array detector (DAD) and spectral analysis from 200 nm to 400 nm wavelengths. A 0.1 mg/mL analytical standard is made from certified reference material for each compound (Cayman Chemical Company, USA). Each sample is prepared by diluting fermentation biomass from a recombinant host expressing the engineered biosynthesis pathway 1:3 or 1:20 in 100% acetonitrile and filtered in 0.2 um nanofilter vials. The retention time and UV-visible absorption spectrum (i.e., spectral fingerprint) of the samples are compared to the analytical standard retention time and UV-visible spectra (i.e. spectral fingerprint) when identifying the olivetolic acid and related compounds mentioned above. Examples of results from the detection of isolated cannabinoid products via fermentation of recombinant host organisms are shown in FIG. 4 and FIG. 6.

### Example 5. Production of Olivetolic Acid (OA) with Expression Alternative OACs (altOACs and/or PKSCs) in a Modified Host Organism

Cyclase genes such as the altOACs and PKSCs described herein can be expressed in a modified Saccharomyces cerevisiae host cell to yield cannabinoid precursors, such as olivetolic acid and divarinic acid. Construction of a modified host cell expressing a cyclase such as the altOACs, and/or PKSCs can be accomplished by transforming a microorganism such as a modified Saccharomyces cerevisiae via chemical transformation of episomal plasmids containing the gene cassettes encoding cyclases such as altOAC and/or PKSCs. Such plasmid transformation protocols are known by those skilled in the art. Such transformation procedures include chemical transformations or electroporation via mixtures of plasmids and the host microorganism.

FIG. 4 depicts the identification of the cannabinoid precursor, olivetolic acid (OA) in a Saccharomyces cerevisiae host cell expressing either recombinant cannabis OAC or an altOAC. Detection of OA in recombinant host cells expressing the cannabis OAC or altOAC were compared against a negative control host cell which did not express cyclases. Cyclase genes expressed in a recombinant host capable of generating cannabinoids precursors were isolated and identified as described in Example 4. Standard yeast fermentation procedures known to those skilled in the art were carried out. Fermentations were carried out over 48 hrs at 30 Celsius shaking at 250 rpm. Fermentation media included components known to those skilled in the art for growing Saccharomyces cerevisiae, including nitrogen and carbon sources to support yeast growth. Such methods have also been described in application 62/914,404. In addition, the fermentation media contained 10 mM phosphate buffer, 10 mM MgCl2, 15 g/L ammonium sulfate, and 4% glucose. Samples were processed as described in Example 4.

### Example 6. Production of Cannabinoids with Expression of a Recombinant Cannabis OAC (csOAC) with and without Alternative OACs (altOACs and/or PKSCs) in a Modified Host Organism

Cyclase genes such as the altOACs and PKSCs described herein can be expressed singly or in combination to yield cannabinoids such as cannabigerolic acid (CBGA). Construction of a modified Saccharomyces cerevisiae with OACs, altOACs, and/or PKSCs can be accomplished by transforming a microorganism such as a modified Saccharomyces cerevisiae via chemical transformation of episomal plasmids containing the gene cassettes encoding cyclases such as altOAC and/or PKSCs. Such plasmid transformation protocols are known by those skilled in the art. Such transformation procedures include chemical transformations or electroporation via mixtures of plasmids and the host microorganism. When the modified host strain expresses downstream genes of the cannabinoid biosynthesis pathway, such as a CBGA synthase, including those genes described in application US21/42090, the cyclase product, stemming from the cyclases disclosed, including the altOAC and/or PKSCs, is consumed by a CBGA synthase to yield the cannabinoid CBGA.

FIG. 6 depicts the quantification of the cannabinoid CBGA generated by expressing cyclases in a Saccharomyces cerevisiae expressing downstream cannabinoid synthases. Cyclase genes expressed in a recombinant host capable of generating downstream cannabinoids yield CBGA, isolated and identified as in Example 4. Modified host organisms either expressed a recombinant cannabis cyclase, csOAC, alone, or in combination with additional PKSC enzymes. Standard yeast fermentation procedures known to those skilled in the art were carried out. Fermentations were carried out over 48 hrs at 30 Celsius shaking at 250 rpm. Fermentation media included components known to those skilled in the art for growing Saccharomyces cerevisiae, including nitrogen and carbon sources to support yeast growth. Such methods have also been described in U.S. patent application Ser. No. 17/068,636. In addition, the fermentation media contained 10 mM phosphate buffer, 10 mM MgCl2, 15 g/L ammonium sulfate, and 4% glucose. Samples were processed as described in Example 4. The cannabinoid CBGA yield was greater with combinatorial expression of alternative cyclases, such as PKSC26 and PKSC27 depicted in FIG. 6. Combinatorial expression of cyclases yielded 1.5 or 2.9× the relative amount of CBGA compared to a single cyclase, a recombinant cannabis cyclase, csOAC.

What is claimed is:

1. An engineered olivetolic acid cyclase (OAC) enzyme derived from a microorganism or a non-Cannabis plant, wherein the enzyme catalyzes cyclization of a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃,

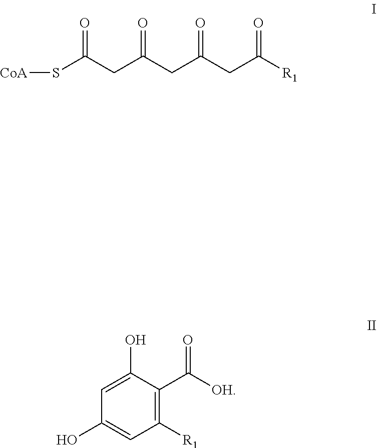

2. The enzyme of claim 1, comprising one or more mutations that increase specificity for particular R₁ alkyl chain length.

3. The enzyme of claim 1, having an amino acid sequence that has less than 50% homology to the sequence set forth in SEQ ID NO: 379.

5. The enzyme of claim 1, having an amino acid sequence that that is at least 95% identical to any one of the sequences set forth in SEQ ID NO: 193-SEQ ID NO: 378.

6. The enzyme of claim 1, having an amino acid sequence that that is at least 50% identical to the sequence set forth in either SEQ ID NO: 385 or SEQ ID NO: 386.

7. The enzyme of claim 1, having an amino acid sequence that that is at least 20% identical to the sequence set forth in SEQ ID NO: 334.

8. The enzyme of claim 1, having an amino acid sequence that that is at least 50% identical to any one of the sequences set forth in SEQ ID NO: 193-SEQ ID NO: 195.

9. An isolated nucleic acid encoding the enzyme of claim 1.

10. The isolated nucleic acid of claim 9, which is codon optimized for production in yeast.

11. The codon-optimized nucleic acid of claim 10, inserted in a vector configured for replication and protein expression in yeast cells.

12. The isolated nucleic acid of claim 9, having a nucleic acid sequence that is at least 95% identical to a sequence selected from the group consisting of SEQ ID NO: 1-SEQ ID NO: 186.

13. An expression cassette comprising the isolated nucleic acid of claim 9.

14. The expression cassette of claim 13, which is a yeast expression cassette.

15. The expression cassette of claim 14, further comprising a nucleic acid fragment at the 5′ end of the isolated nucleic acid, wherein the nucleic acid fragment encodes a codon optimized cofolding peptide having an amino acid sequence that is at least 95% identical to a sequence selected from the group consisting of SEQ ID NO: 380-384.

16. A recombinant microorganism comprising the expression cassette of claim 13, that expresses the engineered OAC enzyme encoded therein.

17. The recombinant microorganism of claim 16, which is a yeast cell, which is a species of Saccharomyces, Candida, Pichia, Schizosaccharomyces, Scheffersomyces, Blakeslea, Rhodotorula, Aspergillus or Yarrowia.

18. The recombinant microorganism of claim 16, further expressing at least one other enzyme in a cannabinoid biosynthetic pathway.

19. The recombinant microorganism of claim 18, wherein the at least one other enzyme is an OAC enzyme having an amino acid sequence that is at least 80% identical to the sequence set forth in SEQ ID NO: 379.

20. The recombinant microorganism of claim 18, wherein the at least one other enzyme is a polyketide synthase or a prenyltransferase.

21. The recombinant microorganism of claim 18, capable of synthesizing a cannabinoid.

22. The recombinant microorganism of claim 18, which is a yeast cell.

23. A method of converting a polyketide (I) into a resorcyclic acid derivative (II), where R₁=CH₃, CH₂CH₃, (CH₂)₂CH₃, (CH₂)₃CH₃, (CH₂)₄CH₃, (CH₂)₅CH₃, or (CH₂)₆CH₃, 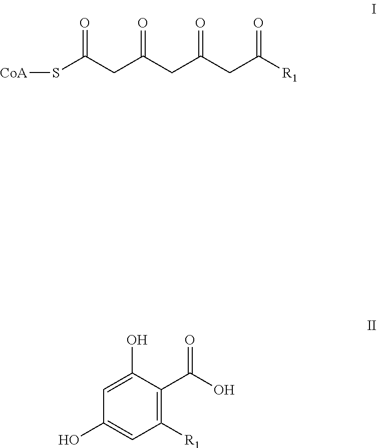
the method comprising contacting the polyketide with the olivetolic acid cyclase (OAC) enzyme of claim 1 in a manner and for a time sufficient to convert the polyketide (I) into the resorcyclic acid derivative (II).

24. The method of claim 23, wherein the olivetolic acid cyclase (OAC) enzyme of claim 1 catalyzes cyclization of the polyketide by a mechanism selected from the group consisting of C2-C7 aldol condensation, Diekmann condensation, Claisen condensation, and Knoevenagel condensation.

25. The method of claim 23, wherein the polyketide and the OAC enzyme are present in the recombinant microorganism of claim 16.

26. The method of claim 25, wherein the recombinant microorganism is the yeast cell of claim 22, and wherein the resorcyclic acid derivative is converted into a cannabinoid in the yeast cell.
